(19) United States
(12) Patent Application Publication
(10) Pub. No.: US 2017/0224423 A1
(43) Pub. Date: Aug. 10, 2017

(54) POSITION DETECTION SYSTEM AND GUIDANCE SYSTEM

(71) Applicant: OLYMPUS CORPORATION, Tokyo (JP)

(72) Inventor: Yusuke SUZUKI, Hino-shi, Tokyo (JP)

(73) Assignee: OLYMPUS CORPORATION, Tokyo (JP)

(21) Appl. No.: 15/498,559

(22) Filed: Apr. 27, 2017

Related U.S. Application Data
(63) Continuation of application No. PCT/JP2016/060226, filed on Mar. 29, 2016.

(30) Foreign Application Priority Data
Jul. 24, 2015 (JP) 2015-146844

(51) Int. Cl. A61B 34/20; G05B 19/04; A61B 1/05; A61B 1/06; A61B 1/00; A61B 1/04

(52) U.S. Cl. CPC A61B 34/20; A61B 1/00158; A61B 1/041; A61B 1/051; A61B 1/00188; A61B 1/0684; G05B 19/041; A61B 2034/2051; A61B 2034/2065; A61M 37/00

(57) ABSTRACT

A position detection system includes: an object to be detected having a magnetic field generator to generate a position detecting magnetic field, and configured to be introduced into a subject; detection coils, each being configured to detect the position detecting magnetic field to output a detection signal; and a processor including hardware. The processor is configured to: correct measured values of detection signals output from the detection coils using a magnetic field generated by applying the position detecting magnetic field to a metal plate, the metal plate being disposed on a side opposite to an area to be detected of the object to be detected relative to the detection coils; and calculate at least one of a position and a direction of the object to be detected using the corrected measured values of the detection signals.

### CROSS REFERENCES TO RELATED APPLICATIONS

This application is a continuation of PCT international application Ser. No. PCT/JP2016/060226 filed on Mar. 29, 2016 which designates the United States, incorporated herein by reference, and which claims the benefit of priority from Japanese Patent Applications No. 2015-146844, filed on Jul. 24, 2015, incorporated herein by reference.

### BACKGROUND

1. Technical Field

The disclosure relates to a position detection system and a guidance system which detect a position and a direction of a capsule medical device introduced into a subject.

2. Related Art

Capsule medical devices have been developed each of which is introduced into a subject to obtain various information about the subject or administer medication to the subject. As an example, a capsule endoscope is known which is formed in a size small enough to be introduced into a subject's digestive tract. The capsule endoscope includes an imaging function and a wireless communication function in a capsule-shaped casing to perform imaging while moving in the digestive tract, after being swallowed into the subject, and wirelessly transmit sequential image data of images of inside an organ of the subject.

Furthermore, a system for performing positional detection has been also developed, using such a capsule medical device as an object to be detected. For example, a position detection system is disclosed in JP 2008-132047 A. The position detection system includes a capsule medical device including a magnetic field generation coil generating a magnetic field upon power supply, and magnetic field detection coils detecting the magnetic field generated by the magnetic field generation coil from outside a subject, and performs calculation for detecting the position of the capsule medical device, on the basis of an intensity of the magnetic field detected by the magnetic field detection coil. Hereinafter, the magnetic field detection coil is simply referred to as detection coil.

### SUMMARY

In some embodiments, a position detection system includes: an object to be detected having a magnetic field generator provided therein to generate a position detecting magnetic field, and configured to be introduced into a subject; a plurality of detection coils disposed outside the subject, each detection coil being configured to detect the position detecting magnetic field to output a detection signal; and a processor including hardware. The processor is configured to: correct measured values of detection signals output from the detection coils using a magnetic field generated by applying the position detecting magnetic field to a metal plate, the metal plate being disposed within a range covering at least opening surfaces of the detection coils, on a side opposite to an area to be detected of the object to be detected relative to the detection coils; and calculate at least one of a position and a direction of the object to be detected using the corrected measured values of the detection signals.

In some embodiments, a guidance system includes: the position detection system including; the object to be detected configured to be introduced into the subject including: the magnetic field generator configured to generate the position detecting magnetic field being an alternating magnetic field having a predetermined frequency; and a permanent magnet provided in the object to be detected; and a guidance device configured to generate a magnetic field acting on the permanent magnet to guide the object to be detected.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic view illustrating an exemplary configuration of a position detection system according to a first embodiment of the disclosure;

FIG. 2 is a schematic diagram illustrating an example of an inner structure of a capsule endoscope illustrated in FIG. 1;

FIG. 3 is a schematic diagram illustrating configurations of a magnetic field detection device and a calculation device illustrated in FIG. 1;

FIG. 4 is a schematic diagram illustrating a position detection method for the capsule endoscope;

FIG. 5 is a flowchart illustrating the position detection method according to the first embodiment of the disclosure;

FIG. 6 is a schematic diagram illustrating a method of calculating correction components for a magnetic field.

FIG. 7 is a schematic diagram illustrating the method of calculating correction components for a magnetic field;

FIG. 8 is a schematic diagram illustrating the method of calculating correction components for a magnetic field;

FIG. 9 is a schematic view illustrating an exemplary configuration of a guidance system according to a second embodiment of the disclosure;

FIG. 10 is a schematic diagram illustrating an example of an inner structure of a capsule endoscope illustrated in FIG. 9; and

FIG. 11 is a schematic diagram illustrating exemplary configurations of a guidance magnetic field generator and a guidance magnetic field controller illustrated in FIG. 9.

### DETAILED DESCRIPTION

A position detection system and a guidance system according to embodiments of the disclosure will be described below with reference to the drawings. Note that, in the embodiments described below, an example of a capsule endoscope orally introduced into a subject to image inside a subject's digestive tract is described according to one aspect of an object to be detected which is to be detected by the position detection system, but the disclosure is not limited to these embodiments. That is, the disclosure can be applied to positional detection of, for example, a capsule endoscope moving in a subject's lumen, from an esophagus to an anus, a capsule medical device delivering medication or the like into a subject, a capsule medical device including a pH sensor measuring the pH in a subject, a marker indicating an irradiation position to which radiation is applied in a radiation examination system, or a marker indicating an irradiation position to which ultrasound is applied in an ultrasonic irradiation system.

Furthermore, in the following description, the drawings merely schematically illustrate shapes, sizes, and positional relationships to the extent that the contents of the disclosure can be understood. Accordingly, the disclosure is not limited only to the shapes, sizes, and positional relationships exemplified in the drawings. Note that, in the drawings, the same portions are denoted by the same reference signs.

### First Embodiment

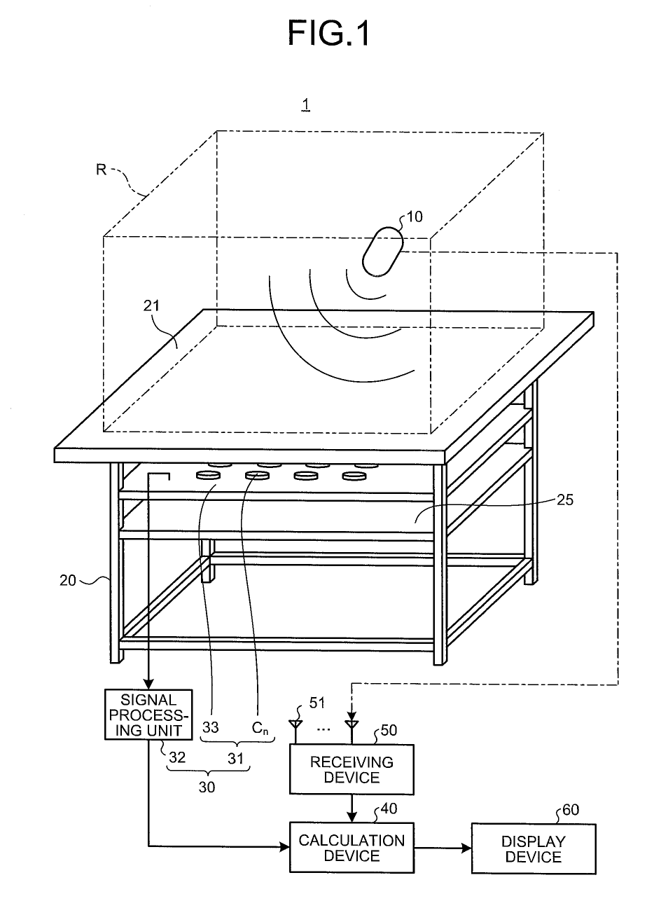

FIG. 1 is a schematic view illustrating an exemplary configuration of a position detection system according to a first embodiment of the disclosure. As illustrated in FIG. 1, a position detection system 1 according to the first embodiment includes a capsule endoscope 10 as an object to be detected generating a position detecting magnetic field, a metal frame 20, a bed 21 supported by the metal frame 20, and positioning thereon a subject into which the capsule endoscope 10 is inserted, a magnetic field detection device 30 disposed under the bed 21, detecting the position detecting magnetic field generated by the capsule endoscope 10, and outputting detection signals, a metal plate 25 disposed between the bed 21 and the magnetic field detection device 30, and a calculation device 40 performing calculation processing for positional detection or the like of the capsule endoscope 10, on the basis of the detection signals output from the magnetic field detection device 30.

In this configuration, the magnetic field detection device 30 has a coil unit 31 including a plurality of detection coils Cₙ detecting the position detecting magnetic field and a panel 33 supporting the detection coils Cₙ, and a signal processing unit 32 performing signal processing on the detection signals output from the detection coils Cₙ. An area in which the position of the capsule endoscope 10 can be detected by the coil unit 31 is an area R to be detected. The area R to be detected is a three-dimensional area including a range in which the capsule endoscope 10 can be moved in the subject, and is set beforehand in a predetermined area on the bed 21. On the basis of the area R to be detected, positions to which the detection coils Cₙ are disposed, or an intensity or the like of the position detecting magnetic field generated by the capsule endoscope 10 are set beforehand.

Furthermore, the position detection system 1 may further include a receiving device 50 receiving a signal wirelessly transmitted from the capsule endoscope 10, and a display device 60 displaying an in-vivo image of the subject captured by the capsule endoscope 10 or positional information or the like of the capsule endoscope 10. In this configuration, the receiving device 50 receives the signal, for example, through a plurality of receiving antennas 51 put on a body surface of the subject.

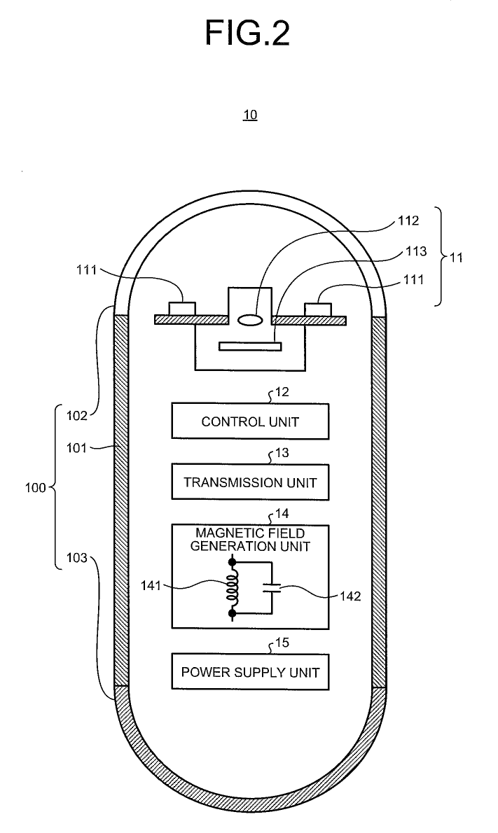

FIG. 2 is a schematic diagram illustrating an example of an inner structure of the capsule endoscope 10 illustrated in FIG. 1. As illustrated in FIG. 2, the capsule endoscope 10 includes a casing 100 having a capsule shape formed into a size small enough to be introduced into the subject, an imaging unit 11 housed in the casing 100, imaging inside the subject, and acquiring an imaging signal, a control unit 12 controlling the operation of respective units of the capsule endoscope 10 including the imaging unit 11, and performing predetermined signal processing on the imaging signal acquired by the imaging unit 11, a transmission unit 13 wirelessly transmitting the imaging signal subjected to the signal processing, a magnetic field generation unit 14 generating an alternating magnetic field as the position detecting magnetic field from the capsule endoscope 10, and a power supply unit 15 supplying power to the respective units of the capsule endoscope 10.

The casing 100 is an outer casing formed in a size small enough to be introduced into a subject's organ. The casing 100 has a cylindrical casing 101 having a cylindrical shape, and domed casings 102 and 103 having a dome shape, and the casing 100 is achieved by closing both opening ends of the cylindrical casing 101 with the domed casings 102 and 103 having a dome shape. The cylindrical casing 101 includes a colored member substantially opaque to visible light. Furthermore, at least one of the domed casings 102 and 103 (the domed casing 102 near the imaging unit 11 in FIG. 2) includes an optical member transparent to light in a predetermined wavelength band, such as visible light. Note that, in FIG. 2, one imaging unit 11 is provided only near the domed casing 102, but two imaging units 11 may be provided, and in this configuration, the domed casing 103 also includes a transparent optical member. Such the casing 100 includes the imaging unit 11, the control unit 12, the transmission unit 13, the magnetic field generation unit 14, and the power supply unit 15 in a liquid-tight manner.

The imaging unit 11 has an illumination unit 111 such as an LED, an optical system 112 such as a condenser lens, and an imaging element 113 such as a CMOS image sensor or a CCD. The illumination unit 111 emits illumination light such as white light to a field of view of the imaging element 113, and illuminates the subject in the field of view, through the domed casing 102. The optical system 112 focuses light reflected from the field of view on an imaging surface of the imaging element 113 to form an image. The imaging element 113 converts the reflected light from the field of view (optical signal) received on the imaging surface, to an electric signal, and outputs the signal as an image signal.

The control unit 12 operates the imaging unit 11 at a predetermined imaging frame rate, and causes the illumination unit 111 to emit light in synchronization with imaging timing. Furthermore, the control unit 12 performs A/D conversion or other predetermined signal processing on the imaging signal generated by the imaging unit 11 to generate image data. In addition, the control unit 12 causes the power supply unit 15 to supply power to the magnetic field generation unit 14 to generate the alternating magnetic field from the magnetic field generation unit 14.

The transmission unit 13 includes a transmitting antenna, and acquires the image data subjected to the signal processing by the control unit 12 and related information to perform modulation processing, and wirelessly transmitting the image data and related information sequentially to the outside through the transmitting antenna.

The magnetic field generation unit 14 includes a magnetic field generation coil 141 partially constituting a resonance circuit to generate a magnetic field from an electric current flow, and a capacitor 142 forming the resonance circuit together with the magnetic field generation coil 141, and the magnetic field generation unit 14 receives power supplied from the power supply unit 15 to generate the alternating magnetic field having a predetermined frequency, as the position detecting magnetic field.

The power supply unit 15 is a power storage unit such as a button battery or a capacitor, and has a switch unit such as a magnetic switch or an optical switch. When the power supply unit 15 is configured to have the magnetic switch, power is turned on and off by a magnetic field applied from outside, and when the power is turned on, the power of the power storage unit is appropriately supplied to component units (the imaging unit 11, the control unit 12, and the transmission unit 13) of the capsule endoscope 10. Furthermore, the power supply unit 15 being turned off stops power supply to the component units of the capsule endoscope 10.

Returning again to FIG. 1, the frame 20 includes a metal such as stainless steel, in consideration of durability against a load of the subject or bed. That is, the frame 20 is a magnetic field generation material (first magnetic field generation material) which may generate a magnetic field by applying the position detecting magnetic field generated by the capsule endoscope 10 to the magnetic field generation material.

The metal plate 25 has a parallel plate shape, and is inserted into a range covering at least opening surfaces of all the detection coils Cₙ, between the frame 20 and the coil unit 31. The metal plate 25 is also a member which may generate a magnetic field by applying the position detecting magnetic field generated by the capsule endoscope 10 to the member. The kind of metal forming the metal plate 25 is not particularly limited.

Here, in the frame 20, an eddy current is generated by applying the position detecting magnetic field generated by the capsule endoscope 10 to the frame 20, and a magnetic field is generated from the frame 20 by the effect of the eddy current. This magnetic field may influence the detection signals of the position detecting magnetic field, output from the detection coils Cₙ, but it is difficult to estimate the influence. In particular, portions of the frame 20 near surfaces disposed on a floor have a complicated shape to ensure strength, and it is further difficult to estimate the influence of the magnetic field generated.

Therefore, in the present first embodiment, the metal plate 25 is positioned at a portion of the frame 20 positioned on the opposite side to the area R to be detected in which the capsule endoscope 10 is to be detected, relative to the plurality of detection coils Cₙ, in particular, between the portions near the surfaces of the frame 20 disposed on the floor, and the plurality of detection coils Cₙ. Thus, when the plurality of detection coils Cₙ are viewed from the capsule endoscope 10, only the metal plate 25 is considered as an interference source influencing the position detecting magnetic field. When only the metal plate 25 having the parallel plate shape is considered as the interference source, and the shape of the metal plate 25 is simplified, the influence of the metal plate 25 on the position detecting magnetic field, that is, an interference magnetic field can be estimated using a simple calculation. Thus, the detection signals can be corrected on the basis of the estimated interference magnetic field.

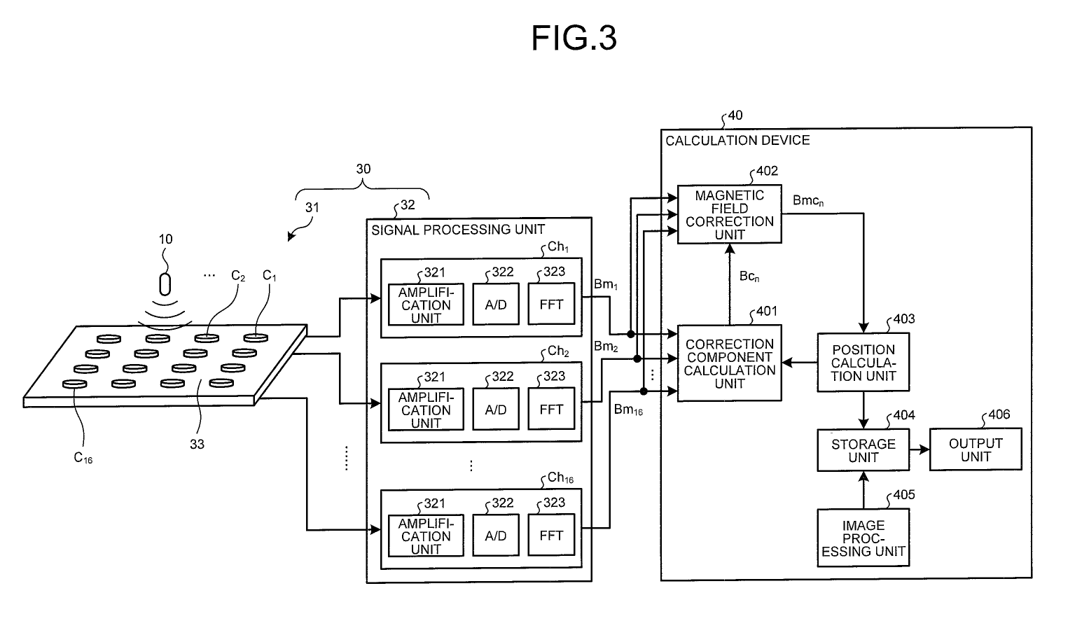

FIG. 3 is a schematic diagram illustrating configurations of the magnetic field detection device 30 and the calculation device 40 illustrated in FIG. 1. The magnetic field detection device 30 includes the coil unit 31 in which the plurality of detection coils Cₙ are arranged, and the signal processing unit 32 processing the detection signals output from the detection coils Cₙ. Here, an index n is a number representing each of the detection coils, and in the case of FIG. 3, n=1 to 16.

Each detection coil Cₙ includes a cylindrical coil obtained by winding a coil wire material into a coil spring shape, and has a size, for example, having an opening diameter of approximately 30 mm to 40 mm, and a height of approximately 5 mm. Each detection coil Cₙ generates an electric current according to a magnetic field distributed at the position of the detection coil Cₙ, and outputs the electric current to the signal processing unit 32, as a detection signal of the magnetic field. These detection coils Cₙ are arranged on a main surface of the panel 33 made of a non-metal material such as a resin and having a flat shape.

The signal processing unit 32 includes a plurality of signal processing channels Chₙ corresponding to the detection coils Cₙ. Each of the signal processing channels Chₙ includes an amplification unit 321 amplifying a detection signal output from a detection coil Cₙ, an A/D converter (A/D) 322 converting the amplified detection signal to a digital signal, and an FFT processor (FFT) 323 performing fast Fourier transformation on the detection signal converted to the digital signal to output a measured value of the detection signal.

The calculation device 40 includes, for example, a personal computer or an all-purpose computer such as a workstation, and performs calculation processing for detecting a position and direction of the capsule endoscope 10, on the basis of the detection signals of the position detecting magnetic field, output from the signal processing unit 32, or calculation processing generating the in-vivo image of the subject on the basis of the image signal received through the receiving device 50.

Specifically, the calculation device 40 includes a correction component calculation unit 401 calculating correction components for the magnetic field which are to be subtracted from the measured values, that is, magnetic field components generated from the frame 20 and the metal plate 25 on the basis of the measured values of the detection signals output from the signal processing channels Chₙ, a magnetic field correction unit 402 subtracting the correction components from the measured values of the magnetic field to calculate true values of the measured values of the magnetic field, and a position calculation unit 403 calculating at least one of the position and the direction of the capsule endoscope 10, on the basis of the corrected measured values.

Furthermore, the calculation device 40 further includes a storage unit 404 storing information or the like about the position and direction of the capsule endoscope 10 calculated by the position calculation unit 403, an image processing unit 405 performing predetermined image processing on the image signal wirelessly transmitted from the capsule endoscope 10 and received by the receiving device 50 (see FIG. 1) to generate image data, and an output unit 406 outputting information about the position and direction or image data stored in the storage unit 404. Hereinafter, information about the position and direction of the capsule endoscope 10 is also simply referred to as positional information.

The storage unit 404 is achieved using a storage medium, such as a flash memory or a hard disk, rewritably storing information, and a reader/writer. The storage unit 404 stores various programs and various parameters for controlling respective units of the calculation device 40, a calculation program for detecting the position of the capsule endoscope 10, and an image processing program, in addition to the positional information or the image data described above.

The receiving device 50 selects a receiving antenna 51 having a maximum reception intensity for a wireless signal transmitted from the capsule endoscope 10, from the plurality of receiving antennas 51 provided outside the subject, and performs demodulation processing or the like on the wireless signal received through the selected receiving antenna 51 to acquire the image signal and the related information.

The display device 60 includes various displays of liquid crystal, organic EL, and the like, and displays the in-vivo image of the subject and the information about the position and direction of the capsule endoscope 10 on a screen, on the basis of the positional information and image data generated in the calculation device 40.

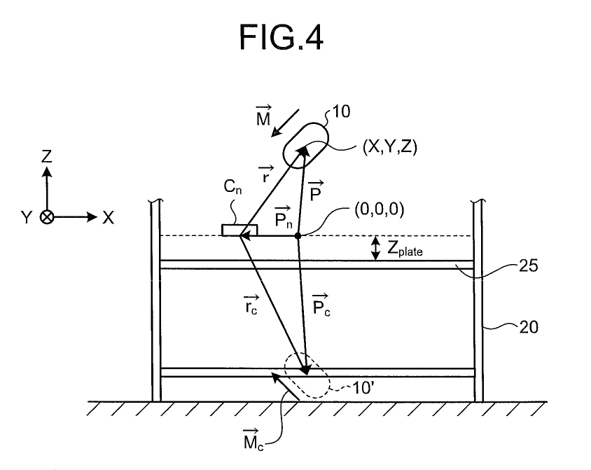

Next, a position detection method according to the first embodiment will be described. FIG. 4 is a schematic diagram illustrating the position detection method for the capsule endoscope 10. In the following description, the origin (0,0,0) is positioned on an arrangement surface on which the detection coils Cₙ are arranged through the panel 33 (see FIG. 1), above the metal plate 25. A distance between the arrangement surface for the detection coils Cₙ and a surface of the metal plate 25 is Zₚₗₐₜₑ.

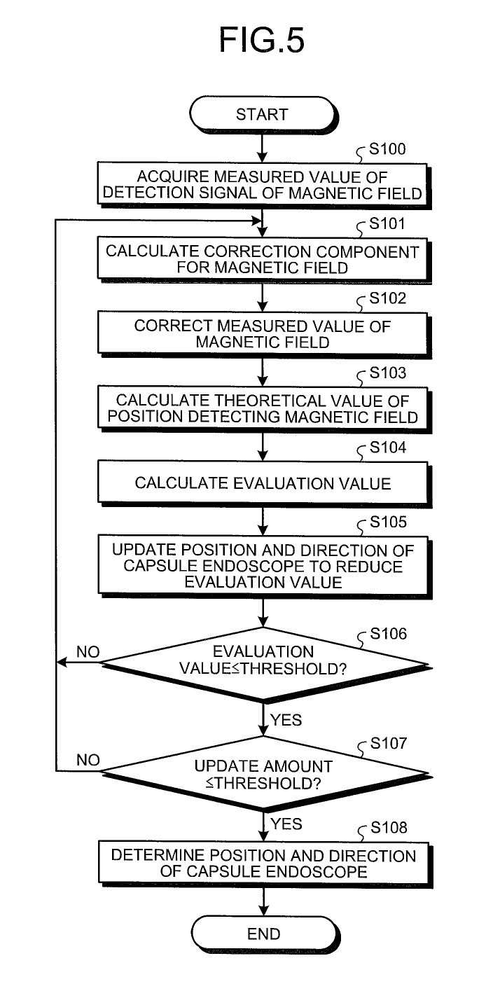

FIG. 5 is a flowchart illustrating the position detection method according to the first embodiment of the disclosure. The flowchart of FIG. 5 illustrates a process of detecting the position detecting magnetic field generated by the capsule endoscope 10 at certain timing by the plurality of detection coils Cₙ, and outputting one set of measured values of the detection signals.

First, in step S100, the calculation device 40 (see FIG. 3) acquires the measured values of the detection signals of the magnetic field detected by the detection coils Cₙ, from the magnetic field detection device 30. Specifically, when each detection coil Cₙ detects the magnetic field and outputs the detection signal, each signal processing channel Chₙ performs amplification, A/D conversion, and FFT processing on the detection signal output from the corresponding detection coil Cₙ, and outputs the detection signal to the calculation device 40. The measured value Bmₙ output from each signal processing channel Chₙ is input to the correction component calculation unit 401 and the magnetic field correction unit 402. These measured values Bmₙ include components of the position detecting magnetic field generated from the capsule endoscope 10, and the magnetic field components generated from the metal plate 25 by applying the position detecting magnetic field to the metal plate 25.

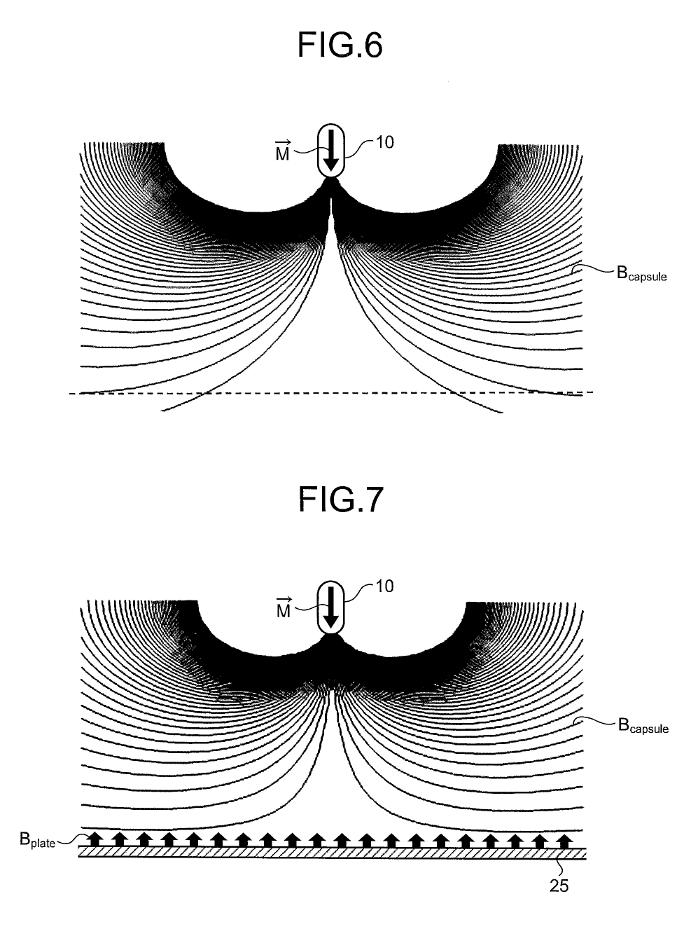

In the subsequent step S101, the correction component calculation unit 401 calculates the correction components for the magnetic field which are to be subtracted from the measured values Bmₙ, on the basis of the measured values Bmₙ obtained in step S100. The correction components correspond to the magnetic field components generated from the metal plate 25 by applying the position detecting magnetic field to the metal plate 25. FIGS. 6 to 8 are schematic diagrams illustrating a method of calculating the correction components for the magnetic field.

FIG. 6 illustrates a magnetic field distribution when there is no metal structure as the interference source in the position detecting magnetic field B_{capsule} generated by the capsule endoscope 10. Note that a vector M illustrated in FIG. 6 indicates a direction of the capsule endoscope 10.

In contrast, FIG. 7 illustrates the metal plate 25 having the parallel plate shape, as the interference source, in the position detecting magnetic field B_{capsule}. In this situation, the magnetic field Bₚₗₐₜₑ is generated from the surface of the metal plate 25 by applying the position detecting magnetic field B_{capsule} to the metal plate 25, and the position detecting magnetic field B_{capsule} is deformed from the influence of the magnetic field Bₚₗₐₜₑ.

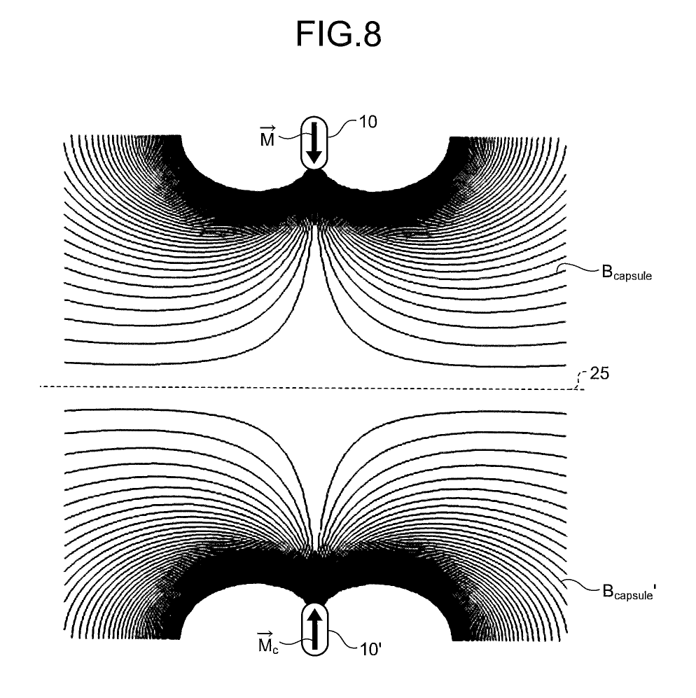

As illustrated in FIG. 8, the deformed position detecting magnetic field B_{capsule} can be considered to be influenced by a magnetic field B_{capsule}′ generated by a magnetic field generation source 10′ similar to the capsule endoscope 10, which is at a position linearly symmetrical to the surface of the metal plate 25. Note that a vector M_{c} illustrated in FIG. 8 indicates a direction of the magnetic field generation source 10′.

A distribution of the magnetic field B_{capsule}′ generated by the magnetic field generation source 10′ is substantially equal to a distribution of the deformed position detecting magnetic field B_{capsule}. Thus, correction is performed to subtract the magnetic field B_{capsule}′ generated by the magnetic field generation source 10′, as a correction magnetic field, from the measured values Bmₙ of the detection signals, and the position detecting magnetic field B_{capsule} (see FIG. 6) without the metal plate 25 as the interference source can be calculated.

When it is assumed that there is the magnetic field generation source 10′, a correction component Bcₙ for correcting each measured value Bmₙ is given by the following formula (1).

$${Bc}_{n} = \frac{μ}{4 π} ( - \frac{{\vec{M}}_{c}}{{\vert {\vec{r}}_{c} \vert}^{3} } + \frac{3 ( {\vec{M}}_{c} \cdot {\vec{r}}_{c} ) \cdot {\vec{r}}_{c}}{{\vert {\vec{r}}_{c} \vert}^{5}} ) \qquad ( 1 )$$

In formula (1), a vector r_{c} is a vector from a detection coil Cₙ to the magnetic field generation source 10′, and is given by the following formula (2), using a positional vector Pₙ of the detection coil Cₙ and a positional vector P_{c} of the magnetic field generation source 10′ relative to the origin (0,0,0), as illustrated in FIG. 4.

\vec{r}_{c}=\vec{P}_{c}−\vec{P}ₙ (2)

In this formula, each component of the positional vector Pc of the magnetic field generation source 10′ is set as follows, on the basis of the latest position (X,Y,Z) of the capsule endoscope 10 determined by the last calculation performed by the position calculation unit 403.

\vec{P}_{c}=(X,Y,−(Z+2Zₚₗₐₜₑ))

Note that when estimation calculation is performed first for the position of the capsule endoscope 10, predetermined initial values are used as the latest position (X,Y,Z) of the capsule endoscope 10.

Furthermore, in formula (1), the vector M_{c} is a directional vector indicating the direction of the magnetic field generation source 10′. When the vector M indicating the direction of the capsule endoscope 10 has components (mₓ,m_{y},m_{z}), components (mₓ,m_{y},−m_{z}) are given for the vector M_{c}.

In the following step S102, the magnetic field correction unit 402 uses the correction component Bcₙ for the magnetic field, calculated in step S101, to correct the measured value Bmₙ of the magnetic field detected by the detection coil Cₙ. A measured value Bmcₙ after correction is given by the following formula (3).

Bmcₙ=Bmₙ−Bcₙ (3)

In the following step S103, the position calculation unit 403 calculates a theoretical value Biₙ of the position detecting magnetic field at a position of each detection coil Cₙ, on the basis of the latest position and direction of the capsule endoscope 10. The theoretical value Biₙ is given by the following formula (4).

$${Bi}_{n} = \frac{μ}{4 π} ( - \frac{\vec{M}}{{\vert {\vec{r}}_{c} \vert}^{3} } + \frac{3 ( \vec{M} \cdot \vec{r} ) \cdot \vec{r}}{{\vert \vec{r} \vert}^{5}} ) \qquad ( 4 )$$

In formula (4), a vector r is a vector from the detection coil Cₙ to an estimation position of the capsule endoscope 10, and is given by the following formula (5), using the positional vector Pₙ of the detection coil Cₙ and a positional vector P of the capsule endoscope 10, relative to the origin (0,0,0), as illustrated in FIG. 4.

\vec{r}=\vec{P}−\vec{P}ₙ (5)

In the following step S104, the position calculation unit 403 calculates an evaluation value S given by the following formula (6), on the basis of the corrected measured value Bmcₙ of the position detecting magnetic field, and the theoretical value Biₙ of the position detecting magnetic field.

$$S = {\sum }_{n = 1}^{16} {\{ {Bmc}_{n} - {Bi}_{n} \}}^{2} \qquad ( 6 )$$

In the following step S105, the position calculation unit 403 updates the position (X,Y,Z) and the direction (mₓ,m_{y},m_{z}) of the capsule endoscope 10 to reduce the evaluation value S.

In the following step S106, the position calculation unit 403 determines whether the evaluation value S is not larger than a threshold of the evaluation value which is set beforehand. For the threshold, a value is set which is small enough to consider that a difference between the measured value Bmₙ and the theoretical value Biₙ of the position detecting magnetic field is within a tolerance. When the evaluation value S is smaller than the threshold, the evaluation value S is determined to be sufficiently small.

When the evaluation value S is larger than the threshold (step S106: No), the process returns to step S101. In this case, in step S101, the position (X,Y,Z) and the direction (mₓ,m_{y},m_{z}) updated in step S105 are used to calculate the correction component Bcₙ.

In contrast, when the evaluation value S is not larger than the threshold (step S106: Yes), the position calculation unit 403 determines whether an update amount Δr in position (X,Y,Z) and an update amount Δm in direction (mₓ,m_{y},m_{z}), which are updated in step S105, are not larger than thresholds set for the position and the direction (step S107). When differences between components of positions before and after update are defined as (ΔX,ΔY,ΔZ), and differences between components of directions before and after update are defined as (Δmₓ,Δm_{y},Δm_{z}), the update amount Δr in position and the update amount Δm in direction are respectively given by the following formulas (7) and (8).

Δr=√(ΔX²+ΔY²+ΔZ²) (7)

Δm=√(Δmₓ²+Δm_{y}²+Δm_{z}²) (8)

Furthermore, for the threshold for determining the update amount Δr and the threshold for determining the update amount Δm, values are set which are small enough to consider that the update amounts Δr and Δm are within a tolerance.

When at least one of the update amounts Δr and Δm is larger than the threshold (step S107: No), the process returns to step S101. In this case, in step S101, the position (X,Y,Z) and the direction (mₓ,m_{y},m_{z}) updated in step S105 are used to calculate the correction component Bcₙ.

In contrast, when both of the update amounts Δr and Δm are not larger than the thresholds (step S107: Yes), the position and the direction updated in step S105 are determined as a position and a direction of the capsule endoscope 10 at that time (step S108). These position and direction are stored in the storage unit 404, as the positional information about the capsule endoscope 10. Then, the process ends.

As described above, according to the present first embodiment, even if there is a magnetic field interference source, such as the frame 20, having an influence on the position detecting magnetic field of the capsule endoscope 10, and it is difficult to estimate the influence, the metal plate 25 inserted between the frame 20 and the detection coils Cₙ facilitates estimation of the influence on the position detecting magnetic field. Thus, the measured values detected by the detection coils Cₙ can be corrected by a simple calculation, and the corrected measured values can be used to accurately detect the position and direction of the capsule endoscope 10.

In the first embodiment described above, the capsule endoscope 10 is described as an example of the object to be detected from which the position and direction are to be detected, but the object to be detected is not limited thereto. For example, in an examination system emitting radiation to a subject to perform examination, a marker indicating an irradiation position to which radiation is applied may be employed as the object to be detected to incorporate the position detection system according to the present first embodiment into this examination system. Alternatively, in an ultrasonic irradiation system, a marker indicating an irradiation position or direction in which radiation is applied may be employed as the object to be detected to incorporate the position detection system according to the present first embodiment into this ultrasonic irradiation system. In any case, when the magnetic field generation unit generating the position detecting magnetic field can be provided in the object to be detected, the position detection system according to the present first embodiment can be applied.

### Second Embodiment

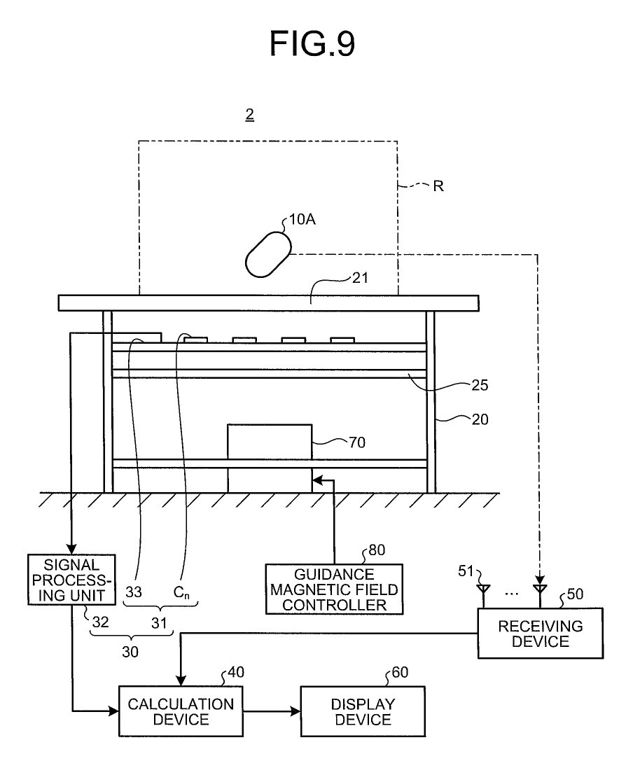

Next, a second embodiment of the disclosure will be described. FIG. 9 is a schematic view illustrating an exemplary configuration of a guidance system according to the second embodiment of the disclosure. As illustrated in FIG. 9, the guidance system 2 according to the present second embodiment includes a capsule endoscope 10A instead of the capsule endoscope 10, and further includes a guidance magnetic field generator 70 and a guidance magnetic field controller 80, in comparison with the position detection system 1 illustrated in FIG. 1. The configurations and operations of the magnetic field detection device 30, the calculation device 40, the receiving device 50, and the display device 60 are similar to those of the first embodiment.

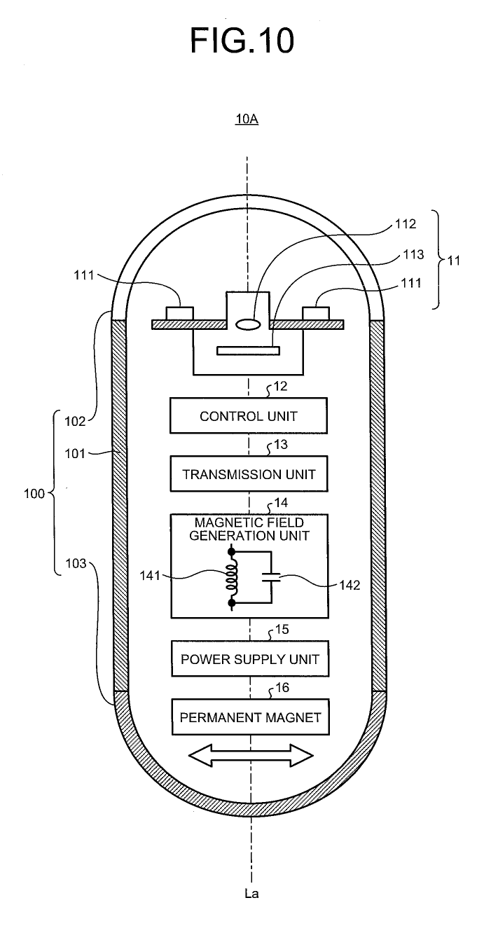

FIG. 10 is a schematic diagram illustrating an example of an inner structure of the capsule endoscope 10A. As illustrated in FIG. 10, the capsule endoscope 10A further includes a permanent magnet 16 in comparison with the capsule endoscope 10 illustrated in FIG. 2. The configurations and operations of the units of the capsule endoscope 10A, other than the permanent magnet 16, are similar to those of the first embodiment.

The permanent magnet 16 is used to guide the capsule endoscope 10A by a magnetic field applied from outside, and is fixedly disposed in the casing 100 so that a magnetization direction is inclined relative to a major axis La of the casing 100. In the present second embodiment, the permanent magnet 16 is disposed so that the magnetization direction is orthogonal to the major axis La as indicated by an arrow. The permanent magnet 16 is operated following the magnetic field applied from outside, and thereby the capsule endoscope 10A is guided by the guidance magnetic field generator 70.

The guidance magnetic field generator 70 and the guidance magnetic field controller 80 generate a guidance magnetic field for changing at least one of a position and a direction of the capsule endoscope 10A introduced into the subject. Here, the direction of the capsule endoscope 10A is represented by inclination (inclination angle) of the major axis La of the capsule endoscope 10A relative to a gravitational axis (Z axis), and a rotation angle (azimuth angle) of the major axis La about the Z axis.

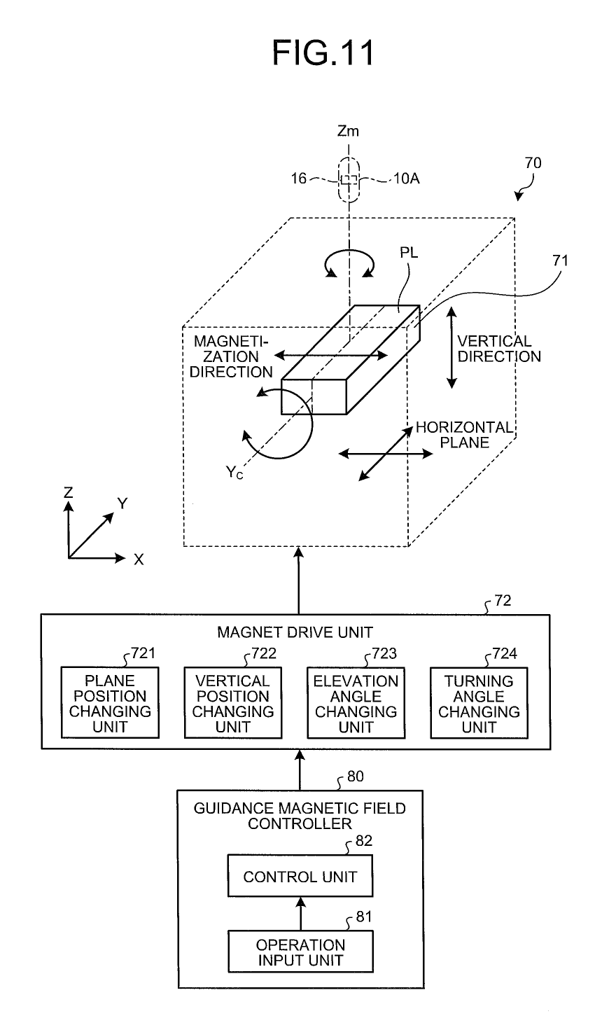

FIG. 11 is a schematic diagram illustrating exemplary configurations of the guidance magnetic field generator 70 and the guidance magnetic field controller 80. As illustrated in FIG. 11, the guidance magnetic field generator 70 includes a permanent magnet (hereinafter, referred to as extracorporeal permanent magnet) 71 generating a magnetic field, and a magnet drive unit 72 changing the position and the direction of the extracorporeal permanent magnet 71. The magnet drive unit 72 of them has a plane position changing unit 721, a vertical position changing unit 722, an elevation angle changing unit 723, and a turning angle changing unit 724.

The extracorporeal permanent magnet 71 includes, for example, a bar magnet having a cuboid shape. In an initial state, the extracorporeal permanent magnet 71 is disposed so that one surface of four surfaces parallel with a magnetization direction of the extracorporeal permanent magnet 71 is parallel with a horizontal plane (plane orthogonal to gravitational direction).

The plane position changing unit 721 is a translation mechanism translating the extracorporeal permanent magnet 71 in a horizontal plane (XY plane). That is, the extracorporeal permanent magnet 71 is moved in the horizontal plane while maintaining a relative position between two magnetized poles in the extracorporeal permanent magnet 71.

The vertical position changing unit 722 is a translation mechanism translating the extracorporeal permanent magnet 71 in a gravitational direction (Z direction). That is, the extracorporeal permanent magnet 71 is moved in a vertical direction while maintaining the relative position between the two magnetized poles in the extracorporeal permanent magnet 71.

The elevation angle changing unit 723 rotates the extracorporeal permanent magnet 71 in a vertical plane including the magnetization direction of the extracorporeal permanent magnet 71 to change an angle of the magnetization direction relative to the horizontal plane. That is, the elevation angle changing unit 723 rotates the extracorporeal permanent magnet 71 about an axis Y_{c} extending in a Y direction, parallel with a capsule facing surface PL, orthogonal to the magnetization direction, and passing through the center of the extracorporeal permanent magnet 71.

The turning angle changing unit 724 rotates the extracorporeal permanent magnet 71 about an axis Zm extending in the Z direction and passing through the center of the extracorporeal permanent magnet 71.

The guidance magnetic field controller 80 includes an operation input unit 81 and a control unit 82. The operation input unit 81 includes an input device, such as a joystick, a console including various buttons or switches, or a keyboard, and inputs a signal according to operation from outside, to the control unit 82. Specifically, the operation input unit 81 inputs, to the control unit 82, an operation signal changing at least one of the position and the direction of the capsule endoscope 10A introduced into the subject, according to the user's operation.

The control unit 82 generates a control signal according to the operation signal input from the operation input unit 81, and outputs the control signal to the guidance magnetic field generator 70.

When the capsule endoscope 10A is guided, the magnet drive unit 72 is driven under the control of the guidance magnetic field controller 80, the extracorporeal permanent magnet 71 is translated in a horizontal or vertical direction, and further the extracorporeal permanent magnet 71 is rotated or turned, thereby changing the direction of the extracorporeal permanent magnet 71. Following such a movement of the extracorporeal permanent magnet 71, the capsule endoscope 10A is guided.

As illustrated in FIG. 9, when the guidance magnetic field generator 70 is provided in the vicinity of the metal plate 25, a non-magnetic metal such as aluminum is used as the metal plate 25. Thus, the influence of the metal plate 25 on the guidance magnetic field is eliminated, and the capsule endoscope 10A can be guided as intended by the user.

Furthermore, the metal plate 25 preferably has a size large enough to cover at least a movable range of the extracorporeal permanent magnet 71 guiding the capsule endoscope 10A. As described above, since the capsule endoscope 10A is moved following the extracorporeal permanent magnet 71, when the movable range of the extracorporeal permanent magnet 71 can be covered by the metal plate 25, influences of the extracorporeal permanent magnet 71 and the magnet drive unit 72 driving the extracorporeal permanent magnet 71 on the position detecting magnetic field can be collected on the metal plate 25, and thus, only the metal plate 25 having the parallel plate shape can be considered as the interference source.

As described above, according to the present second embodiment, even if the guidance magnetic field generator 70 is provided to guide the capsule endoscope 10A, the metal plate 25 is inserted between the guidance magnetic field generator 70 and the detection coil Cₙ, and thus the influence on the position detecting magnetic field can be readily estimated, and the position and direction of the capsule endoscope 10A can be accurately detected.

According to some embodiments, the metal plate is disposed between at least one magnetic field generation material and the plurality of detection coils to correct the measured values of the plurality of detection signals output from the detection coils, using the magnetic field components generated from the metal plate by applying the position detecting magnetic field to the metal plate. Therefore influence of the magnetic field generated by the at least one magnetic field generation material can be inhibited, and accurate positional detection can be performed.

The above first and second embodiments of the disclosure are merely examples for carrying out the disclosure, and the disclosure is not limited to these embodiments. Furthermore, the disclosure may create various embodiments by appropriately combining a plurality of elements disclosed in the first and second embodiments. The disclosure can be modified in various manners in accordance with specifications or the like, and it is obvious from the above description that other various embodiments can be made within the scope of the disclosure.

What is claimed is:

1. A position detection system comprising:
an object to be detected having a magnetic field generator provided therein to generate a position detecting magnetic field, and configured to be introduced into a subject;
a plurality of detection coils disposed outside the subject, each detection coil being configured to detect the position detecting magnetic field to output a detection signal; and
a processor comprising hardware, wherein the processor is configured to:
correct measured values of detection signals output from the detection coils using a magnetic field generated by applying the position detecting magnetic field to a metal plate, the metal plate being disposed within a range covering at least opening surfaces of the detection coils, on a side opposite to an area to be detected of the object to be detected relative to the detection coils; and
calculate at least one of a position and a direction of the object to be detected using the corrected measured values of the detection signals.

2. The position detection system according to claim 1, further comprising
a magnetic field generation material configured to generate a magnetic field by applying the position detecting magnetic field to the magnetic field generation material,
wherein the magnetic field generation material is a metal frame of a bed on which the subject is positioned, and
the metal plate is disposed within the range covering at least the opening surfaces of the detection coils, between the magnetic field generation material and the detection coils.

3. The position detection system according to claim 1, wherein
the processor is configured to:
when it is assumed that there is the object to be detected, which is located at a position symmetrical to the latest position of the object to be detected, relative to the metal plate, calculate, as correction components, values of the detection signals at respective positions of the detection coils in a magnetic field generated by the object to be detected at the position symmetrical to the latest position of the object to be detected, relative to the metal plate; and
perform correction by subtracting the correction components from the measured values of the detection signals.

4. The position detection system according to claim 3, wherein
the processor is configured to:
calculate an evaluation value based on a difference between theoretical values of the position detecting magnetic field assumed to be generated by the object to be detected at a position of the object to be detected determined by the last calculation, and the corrected measured values of the detection signals; and
update at least one of the position and the direction of the object to be detected to reduce the evaluation value.

5. The position detection system according to claim 1, wherein
the object to be detected is a capsule endoscope including an imaging sensor configured to image inside the subject to generate an image signal.

6. A guidance system comprising:
the position detection system according to claim 1 including;
the object to be detected configured to be introduced into the subject including:
the magnetic field generator configured to generate the position detecting magnetic field being an alternating magnetic field having a predetermined frequency; and
a permanent magnet provided in the object to be detected; and
a guidance device configured to generate a magnetic field acting on the permanent magnet to guide the object to be detected.
